(19) United States
(12) Patent Application Publication
(10) Pub. No.: US 2009/0227661 A1
(43) Pub. Date: Sep. 10, 2009

(54) TRANSCRIPTION FACTOR DECOY OLIGODEOXYNUCLEOTIDES HAVING MULTIPLE CIS ELEMENTS

(76) Inventor: Zhiguo Wang, Montreal (CA)

Correspondence Address: Louis Tessier, P.O. Box 54029, Town of Mount-Royal, Quebec, QC, H3P 3H4, CA

(21) Appl. No.: 12/086,852

(22) PCT Filed: Dec. 22, 2006
(86) PCT No.: PCT/CA2006/002125

§ 371 Date: Jun. 20, 2008

Related U.S. Application Data
(60) Provisional application No. 60/752,363, filed on Dec. 22, 2005.

(51) Int. Cl. A61K 31/711; C07H 21/04; C12N 5/06

(52) U.S. Cl. 514/44 R; 536/23.1; 435/375

(57) ABSTRACT

A double-stranded decoy oligodeoxynucleotide, the decoy oligodeoxynucleotide comprising a first strand of deoxynucleotides and a second strand of deoxynucleotides substantially complementary to the first strand of deoxynucleotides, the decoy oligodeoxynucleotide comprising at least two cis elements, each of the cis elements specifically targeting a transcription factor. Also, methods of using the decoy oligodeoxynucleotide.

This application claims priority from U.S. Provisional Patent Applications Ser. No. 60/752,363 filed Dec. 22, 2005, the disclosure of which is hereby incorporated by reference in its entirety.

### FIELD OF THE INVENTION

The present invention relates to decoy oligodeoxynucleotides and their use in gene expression modulation. Specifically, the present invention comprises decoy oligodeoxynucleotides including multiple cis elements each targeting a transcription factor and methods of using same.

### BACKGROUND OF THE INVENTION

Many mammalian diseases are multifactorial and multistep processes. For example, cancer progresses by the accumulation of genetic abnormalities in somatic cells, allowing them to escape from control mechanisms involved in cell differentiation, growth, and death. Targeting a single factor (molecule) may not be adequate and certainly not optimal in cancer therapy because single agents are limited by incomplete efficacy and dose-limiting adverse effects. If related factors are concomitantly attacked, better outcomes are expected. Currently available combination pharmacotherapy was developed for this reason: a combination of two or more drugs or therapeutic agents given as a single treatment successfully saves lives.

The ‘drug cocktail’ therapy of AIDS is one example of such a strategy (Henkel, 1999) and similar approaches have been used for a variety of other diseases including cancers (Charpentier, 2002; Konlee, 1998; Kumar, 2005; Lin et al., 2005b; Nabholtz and Gligorov, 2005; Ogihara, 2003). However, the currently available ‘drug-cocktail’ therapy is costly and may involve complicated treatment regimen, undesired drug-drug interactions and increased side effects as well (Konlee, 1998). There is a need to develop a strategy to avoid these problems and a ‘one drug-multiple targets’ strategy can achieve this goal. However, it is nearly impossible to develop single compounds that have the ability to act on multiple target molecules using traditional pharmaceutical approaches or the currently known antisense strategies.

The decoy oligodeoxynucleotides (dODN) technology of the present invention involves synthetic double-stranded ODNs containing a cis-element with high affinity for a target transcription factor (TF) but with low affinity for non-target TFs. The dODNs can bind the target TF after being introduced into target cells competitively inhibiting endogenous cis-trans interactions, leading to removal of trans-factors from the endogenous cis-element with subsequent modulation of gene expression (Morishita et al., 1995 & 1997). The use of dODN is therefore promising for the treatment of many diseases as it allows for the downregulation of the genes transcribed by the target TFS.

It is known that TFs bind to a cis-element in a cooperative manner, where one molecule of TF binds weakly, but multiple molecules of the same TF engage in protein-protein interactions that increase each of their bindings to the cis-element. To facilitate TF binding to a dODN, one can elevate molar concentration of the dODNs, but this may well elicit toxicity.

In view of the above, there is a need to provide novel methods for targeting diseases and otherwise regulating gene expression by downregulating the action of transcription factors.

### SUMMARY OF THE INVENTION

The invention concerns an innovative decoy oligodeoxynucleotide (dODN) technology in which multiple cis-elements are engineered into single dODNs directed attacking one target TF or multiple target TFs (mimicking a ‘drug cocktail’ approach). The present inventors designed dODNs targeting NF-κB, E2F and Stat3 separately, and complex decoy oligodeoxynucleotides (cdODNs) simultaneously targeting NF-κB, E2F and Stat3. We evaluated the effect of this cdODN on the expression of cancer-related genes, viability of human cancer cell lines and in vivo tumor growth in nude mice. The cdODN targeting NF-κB, E2F and Stat3 together demonstrated more than two-fold enhancement of the efficacy and two orders of magnitude increases in potency, compared with each of the separate dODNs or the combination of all three separate dODNs. The cdODN also showed an earlier onset and longer-lasting action. Most strikingly, the cdODN demonstrated an ability to attack multiple molecules critical to cancer progression via multiple mechanisms, leading to elimination or regression.

In conclusion, it therefore seems that the present invention creates a surprising and unexpected synergy between the multiple cis elements contained in the dODN, whether single or multiple TFs are targeted. Real-time RT-PCR revealed that the cdODNs knocked down expression of the genes regulated by the target transcription factors. The cdODN strategy offers resourceful combinations of varying cis-elements for concomitantly targeting multiple molecules in cancer and other biological processes and opens the door to ‘one drug-multiple targets’ therapy for a broad range of mammalian diseases.

Also, one can engineer multiple consensus sites into one dODN so that one molecule of dODN could provide a number of binding sites for the target TF, even at lower concentrations. For the sake of clarity, we name here the originally defined dODN simplex decoy ODN (sdODN) since it generally contains only one binding site for only one particular TF, and the dODN that incorporates multiple binding sites for one TF (homomeric) or multiple TFs (heterogeneous), complex decoy ODN (cdODN). In the instant application the effects of sdODNs targeting NF-κB, E2F or Stat3 separately and a cdODN targeting the three oncoproteins simultaneously on tumor cell growth and the expression of cancer-related genes were compared. The inventors demonstrate the superiority of the latter over the former.

In a first broad aspect, the invention provides a double-stranded decoy oligodeoxynucleotide, the decoy oligodeoxynucleotide comprising a first strand of deoxynucleotides and a second strand of deoxynucleotides substantially complementary to the first strand of deoxynucleotides, the decoy oligodeoxynucleotide comprising at least two cis elements, each of the cis elements specifically targeting a transcription factor.

In a variant, the decoy oligodeoxynucleotide as defined in claim 1, wherein the cis elements all target specifically the same transcription factor. For example, the decoy oligodeoxynucleotide have cis elements that are substantially identical. In another variant, the cis elements target specifically different transcription factors.

Non-limiting examples of cis elements usable in the present invention includes cis elements cis elements targeting specifically and simultaneously at least two transcription factors selected from the group consisting of: NF-κB, E2F, Stat3, SNAIL, a transcription factor targeting the estrogen receptor responsive element, Brn-3b, SLUG and Ets-binding sites. Other non-limiting examples of cis elements usable in the present invention includes cis elements targeting specifically and simultaneously at least two transcription factors selected from the group consisting of: tumor necrosis factor-alpha, GATA-4, FOG-2 and Janus tyrosine kinase-signal transducer and activator of transcription, Irx5, Irx3 and Etv1.

In some embodiments of the invention, one of the cis elements is comprised in the first strand of deoxynucleotides and another one of the cis elements is in the second strand of deoxynucleotides.

Specific and non-limiting examples of decoy oligodeoxynucleotide as defined in claim 1, wherein one of the first and second strands of deoxynucleotides has the sequence set forth in one of the following SEQ Ids:

GAGUGGGATTTCUCCAGCGTG (SEQ ID No. 04)
TCTAAGTTTCGCGCCCTAGC (SEQ ID No. 05)
GATCCUTTTAGGGAAUCTCCTG (SEQ ID No. 06)

(SEQ ID No. 07)

TCTTGAGUGGGATTTUCCCCAGCCTCTTGAGUGGGATTTCUCCCAG

(SEQ ID No. 08)

TCATAGTTTCUGCGCAAAAUTGAGTTTCGCGCCCTTTC

SEQ ID 09

GATCCUTTCCCGGAAUCTCCTGCCUTTTCGGGAAUCTCCTG

(SEQ ID No. 10)

TCTGAGCUTTCTGGGAAUCTTGGGGACTTTUCGCGCCCTA
TTGCCGTACCTGACTTAGCC (SEQ ID No. 11)

(SEQ ID. No. 12)

TTGCCGTACCTGACTTAGCCTTGCCGTACCTGACTTAGCC

(SEQ ID No. 13)

TCTGAGCUTTCTGAGACUCTTGTGAACTGTUCACGCCCTA.

In some embodiments of the invention, at least one of the cis elements has the sequence set forth in one of the following SEQ IDs

GUGGGATTT (SEQ ID No. 01)
AAAGCGCG (SEQ ID No. 02)
TTTAGGGAA. (SEQ ID No. 03)

In other embodiments of the invention, the decoy oligodeoxynucleotide includes at least three cis elements, the at least three cis elements having respectively the sequences set forth in SEQ IDs NOs: 1, 2 and 3.

In another broad aspect, the invention provides a method for treating or preventing a disease in a mammal, the disease resulting from the increased presence of a specific transcription factor, the method comprising the administration to the mammal of a therapeutically effective amount of a decoy oligodeoxynucleotide, the decoy oligodeoxynucleotide comprising at least two cis elements, each of the cis elements specifically targeting a respective transcription factor, at least one of the cis elements specifically targeting the specific transcription factor. In some embodiments of the invention, the decoy oligodeoxynucleotide is carried in a pharmaceutically acceptable carrier.

A non-limiting example of a disease treatable or preventable using the above method include cancer, such as breast cancer and lung cancer. The cancer may include cancer cells from a cell line selected from breast cancel cell line SK-BR-3, breast cancer cell line MCF-7 and lung cancer cell line A549. Other non-limiting examples of a diseases treatable or preventable using the above method include heart failure and arrhythmia.

In a case wherein the disease is cancer, the method may result in an inhibition in the growth of a tumor.

In another broad aspect, the invention provides a method for reducing tumor growth in a mammal, the method comprising the administration to the mammal of a therapeutically effective amount of a substance simultaneously reducing the expression of transcription factors NF-κB, E2F and Stat3. For example, the substance is administered intra-tumorally.

In some embodiments of the invention, the substance includes a decoy oligodeoxynucleotide comprising a first strand of deoxynucleotides and a second strand of deoxynucleotides substantially complementary to the first strand of deoxynucleotides and bound thereto, the decoy oligodeoxynucleotide comprising at least three cis elements, the at least three cis elements each specifically targeting a respective transcription factor selected from transcription factors NF-κB, E2F and Stat3.

In another broad aspect, the invention provides a method for interfering with the expression of at least two genes in mammalian cells, the expression of each of the at least two genes being regulated by a respective transcription factor, the method comprising the transfection in the cells of a decoy oligodeoxynucleotide, the decoy oligodeoxynucleotide comprising at least two cis elements, each of the cis elements specifically targeting one of the respective transcription factors, the decoy oligodeoxynucleotide being administered in an amount effective for substantially reducing the regulatory action of each of the transcription factors.

In some embodiments of the invention each of the cis element is identical with a target core sequence of the respective transcription factor targeted by the cis element. In a non-limiting example of implementation, each of the cis elements is between 8 and 9 bases long.

In some example of implementation of the above methods, the subject is a non-human mammal or a human.

For the purpose of the present description, strands of deoxynucleotide are substantially complementary to each other if they are stable at their intended use temperature and are able to have a biological effect on their target transcription factors. Thus, they need not be 100% complementary to each other. Cis elements are substantially identical if they are identical to each other or if they have the same effect on their target transcription factor, i.e. the ability to successfully compete with endogenous TF binding sites.

Also, a cis element specifically targets a TF if it produces significant biological effects onto that TF while producing negligible biological effects on other TF. Also, for the purpose of this document, the terminology cis element applies to a sequence of oligonucleotide.

Advantageously, the present invention has the potential to enhance the effect of dODNs on mammalian cells while reducing the toxicity of these dODNs.

In addition, the present invention decreases the delay required to obtain biologically significant effects after the administration of dODNs.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

FIG. 1 illustrates decoy oligodeoxynucleotides (dODNs) sequences designed to specifically target various transcription factors (TFs); the consensus binding sites are in bold and underlined, and the number of consensus binding sites for the specified TF is indicated by the value in the subscript; the substituted nucleotides are indicated by the enlarged letters; for convenience, the negative control are labeled ODNs NC1, NC2 and NC3;

FIG. 2 illustrates the validation of the cdODN technology as an anti-cancer therapeutic approach in an in vitro model using the human breast cancer cell line SKBr-3; shown in panel (A) is a concentration-dependent reduction of cell viability produced by the dODNs: comparisons among the sdODNs, their respective homomeric (also called herein homogenous) cdODNs and heteromeric (also called herein heterogenous) (cdODN (NES); note the downward and leftward shifts of the dose-response curves by cdODNs, relative to the sdODNs, indicating increased maximum effects (Eₘₐₓ) and decreased IC₅₀; symbols are the averaged experimental data and lines represent the fit to the Hill equation; the mean Eₘₐₓ and IC₅₀ values (n=6 independent experiments for each group) are indicated and the values were statistically significant between all cdODNs and sdODNs; the IC₅₀ of NES was significantly smaller than all homogeneous cdODNs; all data points were after normalization with their negative controls (NCs); i.e. sdODNs normalized to NC1 and cdODNs normalized to NC2; for additional control, NC3 was also normalized to NC2. NC1 and NC2 are scrambled ODNs and NC3 is a mutated NES with nucleotide substitution in the core sequences of the cis-elements (see FIG. 1); panel (B): comparison of decreases in cell viability produced by NES and co-transfection of the three sdODNs (N+E+S) as a combination treatment. ‘N+E+S (100 nM)’ indicates a concentration of 100 nM for each sdODN and 300 nM for the total, and ‘N+E+S (33.3 nM)’ indicates a concentration of 33.3 nM for each sdODN and 100 nM for the total; *p<0.05 vs. NES; n=6 independent experiments for each group;

FIG. 3 illustrates the validation of the cdODN technology as an anti-cancer therapeutic approach in an in vitro model using human breast cancer cell line SKBr-3; time-dependent reduction of cell viability produced by the dODNs: comparisons among the sdODNs, the homogeneous cdODNs and the heterogeneous cdODN: data shown were all normalized to the initial values at 0 time point (before ODN transfection); *p<0.05 vs. NC; n=6 independent experiments for each group;

FIG. 4 illustrates the validation of the cdODN technology as an anti-cancer therapeutic approach in an in vivo s.c. model of tumors induced by SKBr-3 cells in nude mice; (A) effects of decoy ODNs on tumor growth as a function time (day) with daily injection of the dODNs; *p<0.05, F-test, NES vs. each of the sdODNs: NF-kB1, E2F1 or Stat31; +p<0.05, F-test, NES vs. N+E+S; (B) comparisons of tumor volumes at day 7 after daily dODN injection between the mice treated with the sdODNs (NF-kB1, E2F1 and Stat31), co-application of the sdODNs (N+E+S) and the cdODN (NES); Eight animals were used for each group *p<0.05 vs. NC2; +p<0.05 vs. each of the sdODNs;

FIG. 5 illustrates the validation of dODNs' ability to bind target transcription factors (TFs) and to manipulate expression of the target genes; (A) EMSA showing the ability of the dODNs to bind the target TFs; the numbers above each lane indicate the number of consensus cis-elements; the arrows indicate the positions of the DNA-protein complexes; NC2: negative control with a scrambled ODN; NEP-P: nuclear extract minus; Cold: in the presence of unlabeled dODNs; Anti-N (p65), Anti-E and Anti-S: treated with the antibodies directed against NF-kB, E2F and Stat3, respectively; and Anti-N/-E, Anti-E/-S and Anti-S/-N: concomitantly treated with two antibodies, as indicated; WT, wild-type NES; MT, mutant NES (FIG. 1); arrows indicate the supershift bands; (B) subcellular localization of transfected the cdODN (NES); The co-staining (yellow) of nuclei with Alexa Flour 488 (green) and propidium iodide (PI, red) indicates the accessibility of the dODNs to the nuclei; upper panels: magnification ×2000; lower panels: magnification ×100; (C) exponential increase in the percentage of cells (n=4 batches of cells) with successful uptake of FITC-labeled phosphorothioate NES, determined by flow cytometry; (D) quantification of intracellular concentration of FITC-labeled phosphorothioate cdODN ([NES]i) in SKBr-3 cells (n=3 batches of cells) treated with extracellular concentrations of NES ([NES]o) of 100 nM and 1 μM, respectively, as a function of time after transfection; (E) quantification of intracellular concentration of NES ([NES]i) in tumor xenografts in nude mice, 3 days and 7 days after daily injections of FITC-labeled phosphorothioate NES into the tumor mass; and

FIG. 6 illustrates the validation of dODNs' ability to bind target transcription factors (TFs) and to manipulate gene expression; (A) real-time RT-PCR quantification of changes of expression of various genes, produced by dODNs (100 nM); shown are mean data (n=5 independent experiments for each group) expressed as relative level of mRNA after normalization to the effects of NC1 or NC2. *p<0.05 vs. NC; concentration-dependent downregulation of the selected genes by NES; shown are mean data from four independent samples; the data for NES and NC3 were first normalized to those for NC2 and then to the lowest concentration of NES or NC3 (0.01 nM), and NC2 data were not normalized its lowest concentration (0.01 nM); All data are expressed as relative level over control non-treated cells.

### DESCRIPTION OF THE PREFERRED EMBODIMENTS

While the experiments described herein concerned the regulation of human cancer cell growth, one of ordinary skilled in the art will readily appreciate that these experiments may be predictive of biological effects in humans or other mammals and/or may serve as models for use of the present invention in humans or other mammals for any other suitable disease or gene activity regulation.

Materials and Methods

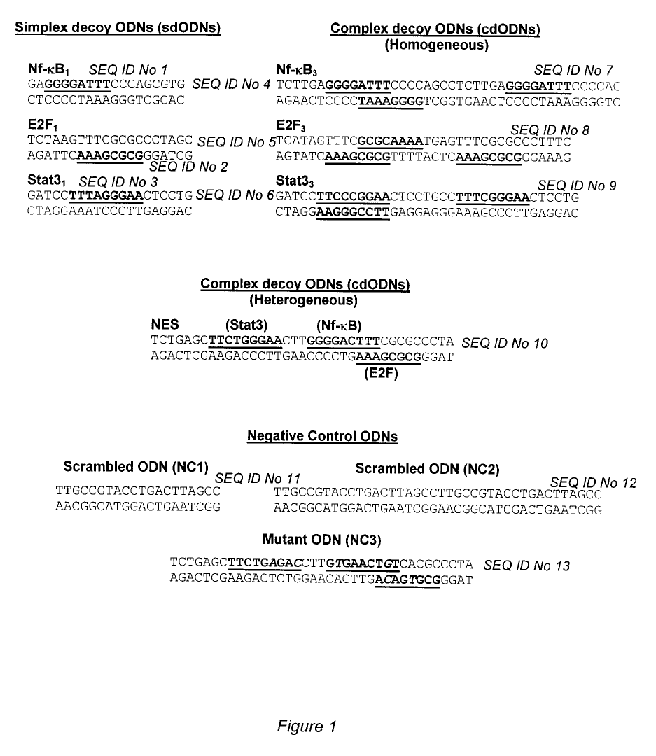

Preparation of decoy ODNs. Single-stranded phosphorothioate oligodeoxynucleotides were synthesized by IDT incorporation (Coralville, Iowa). The ODNs were washed in 70% ethanol, dried and dissolved in sterilized Tris-EDTA buffer (10 mM Tris+1 mM EDTA). The supernatant was purified using Micro Bio-spin30 columns (BioRad, Hercules, Calif.) and quantified by spectrophotometry. The double-stranded dODNs were then prepared by annealing complementary single stranded oligodeoxynucleotides (FIG. 1) by heating to 95° C. for 10 min followed by cooling to room temperature (RT) slowly over 2 h.

Cell culture. The human breast cancer cell lines SKBr-3 and MCF-7 were grown in McCoy's 5a medium, and A549 human lung cancer cells were grown in Ham's F12K medium (Wang et al., 2002). All the cells and media were purchased from the ATCC (Manassas, Va.).

Electrophoresis mobility shift assay (EMSA). The dODNs were labeled by mixing 4 μl (50 ng) annealed dODNs with 4 μl T4 kinase buffer (5×), 1 μl DTT (0.1 M), 6 μl [γ-³²P]-ATP, 3 μl ddH₂O and 2 μl T4 kinase. The sample was incubated at 37° C. for 1 h and then 80 μl TE (10 mM Tris-HCL pH 8.0) was added to complete the reaction. The sample was then loaded into the G-25 column and centrifuged at 7000 g for 2 min. The nuclear extract of human cancer cell lines SKBr-3 was purchased from Santa Cruz Biotechnology (Santa Cruz, Calif.). Binding reactions were carried out at RT for 15 min in a buffer containing 1.2 μg nuclear extracts in 10 μl H₂O and 8 μl of master mix (12×) containing 1 M Tris-HCl (pH7.5), 0.5 M EDTA, 5 M NaCl, 1 M DTT, 50% glycerol, 100 mg/ml BSA, and 1 mg/ml poly dIdC. For super shift experiment, antibodies (1 μg; anti-NF-κB/p65 antibody from Santa Cruz, and anti-E2F and STAT3 from Cell Signaling) were included in the reaction, and for competition experiments, unlabeled dODNs in 100-fold excess of the labeled dODNs were added in the binding reactions. Then, 2 μl (100,000 CPM/μl) ³²P-labeled dODNs were added to the reaction and incubated for another 15 min at RT, followed by addition of 2 μl loading dye. DNA-protein complexes were separated by non-denaturing polyacrylamide gel (7.5% in 0.4×TBE) electrophoresis. Gels were dried and analyzed with Typhoon image system (Amersham Bioscience) and quantified with ImageQuant software (Version 5.2).

Decoy ODN transfection. The cells were transfected with different concentrations of dODNs using Lipofectamine 2000 (Invitrogen, Carlsbad, Calif.). For viability studies, cells were seeded in 96-well tissue culture plates. At 50% confluence, the cells were washed with serum-free medium once and then incubated with 50 μl fresh fetal bovine serum (FBS)-free medium. Decoy ODNs of varying concentrations and lipofectamine (0.25 μl) were separately mixed with 25 μl of Opti-MEM® I Reduced Serum Medium (Gibco, Grand Island, N.Y.) for 5 min. Then the two mixtures were combined and incubated for 20 min at RT. The lipofectamine:dODNs mixture was added dropwise to the cells and incubated at 37° C. for 5 h. Subsequently, 25 μl fresh medium containing 30% FBS was added to the well and the cells were maintained in the culture until use, either for cell growth assays or for RNA extraction.

Subcellular localization of transfected dODNs. The dODNs were labeled with Alexa Flour 488 using ULYSISP®P Nucleic Acid Labeling kits (Invitrogen). The labeled dODNs were purified with micro Bio-spin30 columns (BioRad). The cells grown on sterile coverslips in 12-well plate were transfected with the dODNs, as described. At the selected post-transfection time points, the cells were washed twice with phosphate-buffered saline (PBS) and fixed with 2% paraformaldehyde for 20 min. In order to visualize nuclear DNA, the fixed cells were equilibrated in 2×SSC solution (0.3 M NaCl, 0.03 M sodium Citrate, pH 7.0) and incubated with 100 μg/ml DNase-free RNase in 2×SSC for 20 min at 37° C. The sample was rinsed three times in 2×SSC and incubated with 5 μM propidium iodide (PI, Invitrogen) for 30 min at RT. The coverslips were mounted onto slides with DABCO medium. The samples were examined under a laser scanning confocal microscope (Zeiss LSM 510) with Alexa Flour 488 (excitation at 492 nm and emission at 520 nm) or with PI (excitation at 535 nm and emission at 617 nm). The images were analyzed by Zeiss LSM software suite.

Real-time RT-PCR. RNA was isolated with RNeasy® Mini Kit (Qiagen), according to the manufacturer's protocols and treated with DNase I to remove genomic DNA. TaqMan quantitative assay of transcripts was performed with real-time two-step reverse transcription PCR (GeneAmp 5700, PE Biosystems), involving an initial reverse transcription with random primers, as previously described (Pang et al., 2003). Human GAPDH control reagents (Applied Biosystems) were used as internal controls.

Determination of cell viability. Cell viability was determined by three methods, as previously described in detail (Wang et al., 2002; Ji et al., 2004; Lin et al., 2005b). In the first method, cells were seeded in 96-well tissue culture plates. At 50% confluence, the growth of cells was synchronized in defined serum-free medium for 5 h. The cells were then transfected with decoy ODNs as described above. Sixteen hours later, the cells were washed with PBS, harvested by trypsinization and suspended in 100 μl medium. Ten μl cell suspension was used for manual counting using hemacytometer (Sigma-Aldrich, Horsham, Pa.) and the counting for each sample was performed in duplicate.

In the second method, cell proliferation was assessed by characterizing the log phase growth with population doubling time (PDT) calculated by the equation: 1/(3.32×(log N_{H}−log N_{I})/(t₂−t₁), where N_{H} is the number of cells harvested at the end of the growth period (t₂) and N_{H} is the number of cells at 5 h (t₁) after seeded (Wang et al., 2002).

The third method used to determine cell viability in our study was the WST-1 kit (Roche, Penzberg, Germany). Briefly, 18 h after treatment with dODNs, cells were washed with PBS and grown in 100 μl fresh culture medium plus 10 μl WST-1 reagent for 30 min. The absorbance was measured at 425 nm using a Spectra Rainbow microplate reader (TECAN, Austria) with a reference wavelength of 690 nm.

Subcutaneous (s.c.) tumor xenografts and assessment of growth. The procedures were similar to previously described (Ji et al., 2004). Four-week-old female BALBc nu/nu nude mice (Charles River Laboratories, Willington, Mass.) were housed five/cage in a pathogen-free environment Punder controlled conditions of light and humidity in the Animal House of Harbin Medical University on a standard sterilizable laboratory diet. Mice were quarantined one week before experimental manipulation and at the end of the quarantine SKBr-3 cells (5×10⁶) were inoculated subcutaneously (s.c.) to the left dorsal flank of mice. When tumor size reached ˜50 mm³ (about 7 days post-inoculation), animals were randomly divided into five groups and NF-κB1, E2F1, Stat31, a scrambled ODN, separately, co-application with all three sdODNs or NES was administered daily by a single intratumoral injection (20 μl of 100 nM dODNs mixed with lipofectamine-2000). Tumor growth was monitored regularly and the volumes (V) of tumors at day 7 after dODN treatment was calculated using the formula V=1/2×length×(width)². All operative procedures and animal care strictly conformed to Guidelines set by the Animal Ethics Committee of the Harbin Medical University.

Measurement of uptake of fluorescent dODNs. One day before treatment, SKBr-3 cells were plated in 24-well format with 1×10⁵ cells/well in 500 μl. On the day of treatment, the cells were incubated with FITC-labeled phosphorothioate ODNs (100 nM and 1 μM), in the presence of Lipofectamine 2000 for 4 h. Following incubation, the cells were harvested with PBS-EDTA and washed twice with PBS, and then soaked in TBS+50 mM glycine (10 min). The amount of internalized phosphorothioate ODNs was determined by flow cytometry. The concentration of ODNs associated with SKBr-3 cells was estimated by interpolation from a standard curve of known FITC (Molecular Probes, Eugene, Oreg.).

For measurement of intracellular cdODN concentration in tumor cells from the nude mice, xenograft pieces were dissected from the animals injected with NES (100 nM):lipofectamine mix for 3 days and 7 days. The preparation was minced and then digested with 0.5 mg/ml collagenase type IV (SIGMA Chemical Co) at 37° C. Cells were dispersed by trituration and washed three times with PBS. The amount of cell-associated phosphorothioate ODNs was determined by flow cytometry, as described above.

Control experiments. For all experiments, negative control (NC) was performed with NC1, NC2 or NC3 ODNs, as shown in FIG. 1. Specifically, for experiments involving sdODNs, NC1 was used and for those with cdODNs, NC2 was used. Additional control was carried out with NC3 as specified. The data presented were all normalized to their respective NCs.

Statistical analysis. Group data are expressed as mean±S.E. Statistical comparisons (performed using ANOVA followed by Dunnett's method) were carried out using Microsoft Excel. A two-tailed p<0.05 was taken to indicate a statistically significant difference. Nonlinear least square curve fitting was performed with GraphPad Prism.

Results

Design of the cdODN. We designed a series of dODNs with consensus sequences for NF-κB (GUGGGATTT: SEQ ID NO 1), E2F (AAAGCGCG: SEQ ID NO 2) and Stat3 (TTTAGGGAA: SEQ ID NO 3) (FIG. 1), and evaluated the effects of these dODNs on gene transcription, tumor cell growth and in vivo tumor growth. For convenience, we labeled the sdODNs containing only one cis-element as follows: NF-κB1, E2F1 and Stat31. The cdODNs containing three identical cis-elements were named NF-κB3, E2F3 and Stat33. We also integrated the cis-elements for NF-κB, E2F and Stat3 together into one heteromeric cdODN molecule which we designated NES. The criteria for selecting these oncoproteins for targeting were two-fold. First, these TFs play critical roles in cancer generation and progression and the feasibility of dODNs targeting these TFs as therapeutic agents for human cancers have been documented (Ahn et al., 2003; T Chan et al., 2004; Dolcet et al., 2005; Leong et al., 2004; Mann et al., 1999; Xi et al., 2005; Yokoyama et al. 2005). Use of these TFs should facilitate the comparison between the sdODNs and cdODN. Second, more importantly, we aimed to attack concomitantly multiple processes determining tumorigenesis and cancer progression. NF-κB is known to antagonize apoptosis and promote cell proliferation, E2F is the major factor for the regulation of cell cycle progression, and cumulative evidence supports a role for aberrant Stat3 activation in transformation and tumor progression partly due to its anti-apoptotic effects via repression of p53. By removing the trans-actions of these TF oncoproteins, one expects to produce a strong anti-proliferation and proapoptotic, and thus anti-cancer, profile.

The rules for designing cdODNs were set to ensure high affinity, high specificity and short length principles. First, each of the cis-elements contained 100% homology to the consensus core sequences for the target TFs and optimal matrix similarity (optimal similarity with base pairs adjacent the consensus core sequence). Second, multiple cis-elements were organized in a way that should produce the least non-specific binding to non-targeted TFs and this is one of the reasons that the E2F cis-element was placed on the antisense strand of the cdODN (NES, FIG. 1). And third, the length of cdODNs was limited to as short as possible without affecting TF binding because short ODNs might be easier to enter into the cell and nucleus and should have less chance to allow for binding by non-target TFs. This is another reason why in NES design, the cis-elements were arranged into both the sense and antisense strands of our NES cdODN. We used the Genomatix™ software available from http://www.genomatix.de/company to predict the specificities towards their respective TFs to obtain optimal organization and length of multiple cis-elements in one cdODN. Their ability to bind with the target TFs was examined by electrophoresis mobility shift assay (EMSA) as to be described in a later section. Scrambled and mutated dODNs were also designed for negative control experiments (FIG. 1)

The following sequences were used:

SEQ ID No. 04

sdODN for NF-_{κ}B1: GAGUGGGATTTCUCCAGCGTG

SEQ ID No. 05

sdODN for E2F: TCTAAGTTTCGCGCCCTAGC

SEQ ID No. 06

sdODN for Stat3: GATCCUTTTAGGGAAUCTCCTG

SEQ ID No. 07

sdODN for NF-_{κ}B1:
TCTTGAGUGGGATTTUCCCCAGCCTCTTGAGUGGGATTTCUCCCAG

SEQ ID No. 08

sdODN for E2F:
TCATAGTTTCUGCGCAAAAUTGAGTTTCGCGCCCTTTC

SEQ ID No. 09

sdODN for Stat3:
GATCCUTTCCCGGAAUCTCCTGCCUTTTCGGGAAUCTCCTG

SEQ ID No. 10

cdODN:
TCTGAGCUTTCTGGGAAUCTTGGGGACTTTUCGCGCCCTA

SEQ ID No. 11

Negative control 1: TTGCCGTACCTGACTTAGCC

SEQ ID No. 12

Negative control 2:
TTGCCGTACCTGACTTAGCCTTGCCGTACCTGACTTAGCC

SEQ ID No. 13

Mutant ODN:
TCTGAGCUTTCTGAGACUCTTGTGAACTGTUCACGCCCTA

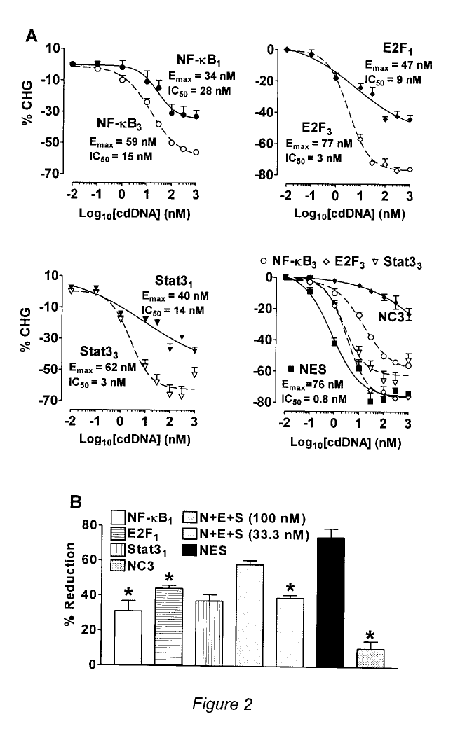

Concentration-dependence of anti-growth effects. To examine the concentration-dependence of anti-growth effects, we evaluated the effects of cdODNs on viability of SKBr-3 human breast cancer cells, as compared with those of the sdODNs (FIG. 2), since SKBr-3 cells have been shown to express the target oncoproteins (Li et al., 2004; Lun et al., 2005). The cdODNs produced concentration-dependent abrogation of cell numbers, as determined 18 h after transfection of dODNs (see the next section for time-dependence of the effects). Both the homomeric (carrying multiple identical cis-elements) and heteromeric (carrying multiple distinct cis-elements) cdODNs demonstrated remarkably greater efficacies and potencies of actions in suppressing cell growth than the sdODNs. These were reflected by the downward shifts of dose-response curves with cdODNs relative to with sdODNs. Specifically, the cdDNAs had nearly 2-fold greater maximum effects than sdDNAs, and even greater intensification (3-fold) was found with NES. The negative controls with scrambled ODNs (NC1 and NC2) did not produced any changes, but NC3 (the mutated NES with nucleotides substitutions) elicited slight depression of cell growth (p>0.05).

The IC₅₀ was reduced by one order of magnitude with the homomeric cdODNs compared with their relative sdODNs, and NES further reduced the IC₅₀ value by another order of magnitude to the pico-molar concentration range (FIG. 2). This is particularly important because the scrambled ODN for negative control (both NC1 and NC2, FIG. 1) also demonstrated non-negligible though not statistically significant decreases in gene expression (10%) and cell viability (15%) at 1 μM, suggesting non-specific and toxic actions of the dODNs at higher concentrations. And by reducing the IC₅₀ from several orders of magnitude of nano-moles with the sdODNs close to the potential toxic concentrations down to 0.8 nM with NES, ˜1000-fold lower than the line, the heterogeneous cdODN should have substantially smaller toxicity.

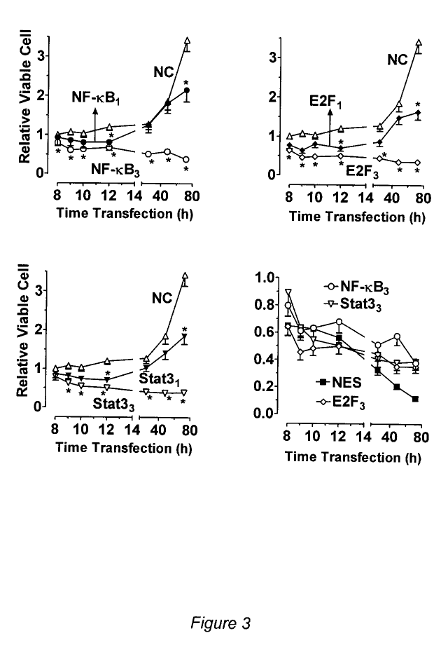

Time-dependence of anti-growth effects. In addition to concentration-dependence, the advantages of cdODNs over sdODNs were also revealed by the time-dependence of the effects (FIG. 3). First, the onset of effects with the cdODNs was much earlier than with the sdODNs; significant diminishment of cell viability took place with cdODNs at around 8 h after transfection, well ahead of that with sdODNs which occurred 12 h after transfection. The effects of sdODNs were bi-phasic, showing initial time-dependent diminishment of cell viability within 18 h and subsequent time-dependent revitalization up to 72 h after transfection. By comparison, the effects of the homogeneous cdODNs reached the maximum or steady-state levels within 10 h. In sharp contrast, the reduction of cell viability in the cells treated with NES developed continuously throughout 72 h period and became virtually non-revivable, leading to complete elimination of the cancer cells. These results indicate that simultaneously attacking multiple targets (NF-κB, E2F and Stat3) remarkably enhances anti-cancer effects as compared with attacking only one target (NF-κB, E2F or Stat3). This point was further evidenced by the fact that effects produced by combination treatment via co-transfection of NF-κB1, E2F1 and Stat31 (100 nM for each) were somewhat smaller than those by NES (100 nM) (FIG. 2, panel B). It must be noted that the total concentration of the combination treatment was 300 nM, 3-fold higher than NES, further suggesting the superiority of cdODNs over sdODNs. Improved effects with cdODNs are presumably a result of increased affinity of binding to TFs and enhanced stability of protein-protein thereby DNA-protein interactions and of increased target versatility. The data presented above are from manual counting of the viable cells and the results were confirmed by modified MTT and flow cytometry methods (data not shown). All these results were consistently reproduced in two other cancer cell lines: A549 and MCF-7, human lung and breast cancer cells, respectively (data not shown).

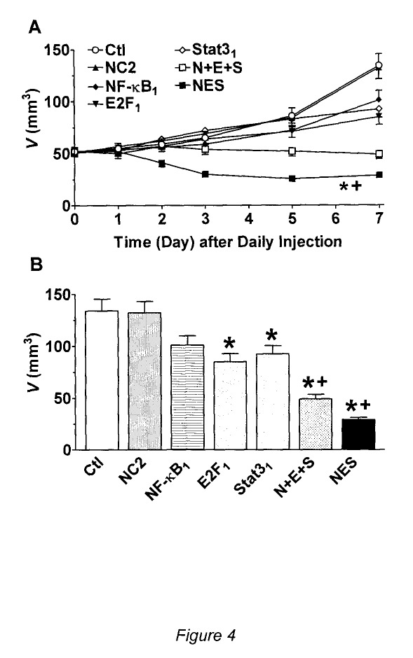

In vivo anti-tumor effects. To further test the dODN technology, we tested the effectiveness of the cdODNs in inhibiting in vivo tumor growth in nude mice. The mice began to receive daily intratumoral injection of lipofectamine 2000-treated ODNs from 7 days after subcutaneous inoculation of SKBr-3 cells and the volume of tumors were measured at fixed time points up to 7 days after drug administration. As depicted in FIG. 4, panel A, the growth of tumors was retarded with the sdODNs NF-κB1, E2F1, Stat31 alone, relative to administration of a scrambled control ODN (NC2). With combination therapy (co-injection of all three different sdODNs), the tumors failed to grow and appeared to stabilize. Most strikingly, application of NES resulted in destruction of the tumors; the tumor mass shrunk to a smaller size than what it was before drug treatment. FIG. 4, panel B demonstrates that the inhibitory effects on tumor growth were clearly in the order of NES>co-transfection (N+E+S)>>NF-κB1, E2F1 or Stat31 alone. For instance, NES caused ˜78% diminishment of tumor volumes, compared to ˜35% and ˜63% decreases produced by E2F1 and N+E+S, respectively.

Potential mechanisms underlying the anti-tumor effects of cdODNs. In order to investigate whether the efficacy of the dONDs is attributable to TF “decoy” effects and not due to non-specific cytotoxicity, the following steps were taken. First, the ability of the dODNs to specifically interact with their corresponding TFs was verified by EMSA in conjunction with super-shift methods (FIG. 3, panel A) using antibodies directed against NF-κB (p65), E2F and Stat3, respectively, with the nuclear extract from SKBr-3 cells. The binding of NF-κB demonstrated clear super-shift. Although the band shift was not seen with E2F and Stat3 antibodies, the DNA bands signals were significantly decreased, indicating the specific bindings of the dODNs with their target proteins. As expected, the cdODNs demonstrated remarkably greater, i.e. synergistic, binding with their respective TFs than the sdODNs, as determined by quantification of the bands using ImageQuant software. For example, the band density with NF-κB3 was 4450±108 (pixel), 18 times greater than that with NF-κB1 (240±18); similarly, E2F3 was ˜12 times greater than E2F1 and Stat33 was 10 times greater than Stat31. NES simultaneously binds to all three target TFs (NF-κB, E2F and Stat3), as indicated by the alternate uses of the antibodies (FIG. 3, panel A).

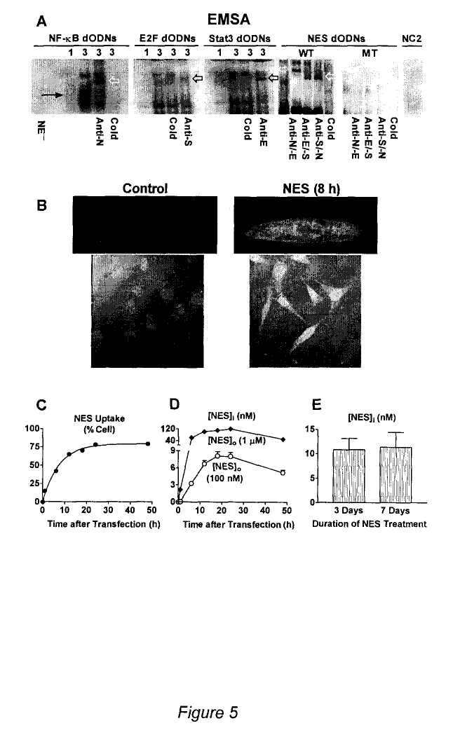

We then went on to confirm the efficiency of transfection and the delivery of these cdODNs into the nuclei of cells (FIG. 3, panel B). The accessibility of the cdODNs into their major site of action is clearly indicated by the overlapping (yellow) of dODN (green) and nucleic (red) fluorescence stainings localized to the nuclei. The nucleic staining did not appear until 6 h after transfection, which is consistent with the time course of the cdDNAs on cancer cell growth. The percentage of cells with successful taking-up of dODNs was similar between the sdODNs (46%) and the cdODNs (42%), determined by counting of cells with clear yellow staining. With time, the percentage of uptake increased. Twenty hours after, the uptake reached up to 79% and it took around 18 h to reach the plateau level. To further verify the uptake of cdODN into the cell, we measured the amount of cell-associated NES with FITC-labeled phosphorothioate NES at two different extracellular concentrations ([NES]o=100 nM and 1 μM). The data are shown in FIG. 5, panel D, where the intracellular concentration of NES ([NES]i) is plotted as a function of time after transfection. The [NES]i reached a maximum level within approximately 18-24 h and the peak [NES]I was 8.1±0.7 nM in the presence of 100 nM [NES]o and 119.5±11.0 nM in the presence of 1 mM [NES]o, equivalent to ˜8% and ˜12% of the 100 nM and 1 mM [NES]o, respectively. The [NES]i in tumor cells isolated from the xenografts of nude mice was also measured at two time points: 3 days and 7 days after daily injection of NES at 100 nM (or [NES]o=100 nM). As shown in FIG. 5, panel E, administration of NES to tumor mass for 3 days yielded an [NES]i 10.8˜2.3 nM and for 7 days an [NES]i 11.3±3.1 nM. The data indicate that daily injection of 100 nM NES created a stable [NES]i which is comparable to the peak level reached by a single application to SKBr-3 cells.

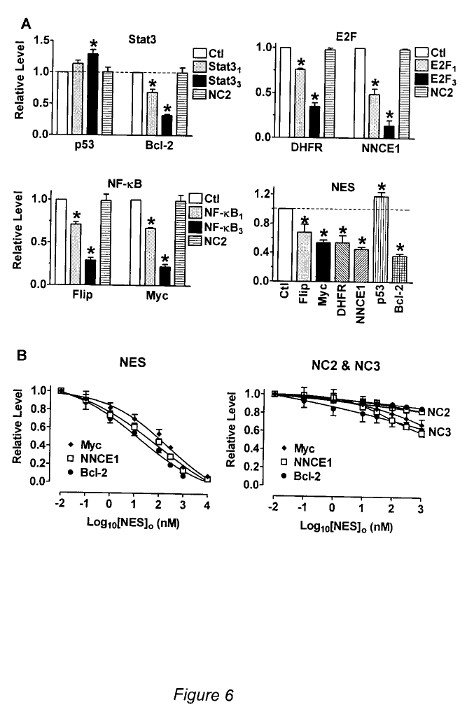

We subsequently studied the gene interference of the dODNs (100 nM) by quantifying mRNA levels with real-time RT-PCR 18 h after transfection and the genes studied included Flip (Kreuz et al., 2001; Micheau et al., 2001) and Myc for NF-κB (Duyao et al., 1992; La Rosa et al., 1994), DHFR (Fry et al., 1999; Park et al., 2003) and CCNE1 (Yasui et al., 2003; Stanelle et al., 2003) for E2F, and p53 (Niu et al., 2005;) and Bcl-2 (Nielsen et al., 1999; Lin et al., 2005a) for Stat3 (FIG. 3, panel C). The dODNs knocked down the transcription of their respective genes. Stat3 dODNs up-regulated p53 transcription, and so did NES. Strikingly, the cdODNs consistently produced more pronounced effects on the transcription than the sdODNs. For example, NF-κB1 and NF-κB3 reduced Myc mRNA levels by ˜13% and ˜48%, respectively. Also notable is that the heterogeneous cdODN NES affected transcription of all the genes examined in this study which are regulated by NF-κB, E2F and Stat3, respectively (FIG. 6, panel A). It should be noted that overall, the expression down-regulation in this study is less than in many previous studies using dODNs; this is because the concentration used in this study is lower (100 nM) than in most of the other studies that generally used >1 mM which could well elicit cytotoxicity in our conditions. To test this notion, we conducted experiments on concentration-dependent downregulation by NES of three selected target gene Myc, NNCE1 and Bcl-2. As illustrated in FIG. 6, panel B (left side), the extent of expression depression of the target genes was increased with increasing [NES]o and at 10 μM the gene expression was virtually abolished. For negative controls, concentration dependence of NC2 (scrambled ODN) and NC3 (mutant NES) on expression of the same set of genes was also studied. As shown in FIG. 6, panel B (right side), NC2 produced minimal effect on gene expression and NC3 elicited certain degrees of gene expression inhibition but the effects did not reach statistical significance (p>0.05).

Discussion

We show here the superiority of simultaneously targeting multiple oncoproteins over targeting single oncoproteins in inhibiting tumor cell growth under both in vitro and in vivo conditions, in terms of the efficacy, potency, toxicity and duration of actions. In addition, heteromeric cdODN show a synergistic effect as compared to the use of multiple homomeric dODNs, which result in improved potency and efficiency. It is known that the ability of CpG-containing oligos to stimulate the innate immune system can yield antitumor efficacy in xenograft tumor models (Kandimalla et al., 2003; Sato et al., 1999). We have analyzed the CpG content of our decoy ODNs and found that none of the dODNs contains the PuPuCGPyPy motif. Hence, the in vitro effects of the dODNs, both phenotypically and molecularly, are consistent with the in vivo antitumor efficacy, neither do the measurements of biological activity strictly correlate with the CpG content of any of the dODNs. The advantages of cdODN are likely ascribed to simultaneous interference of expression of multiple genes controlled by the target TFs. The beauty of this cdODN “one drug-multiple targets” strategy is that it can be used to target one TF using a decoy containing multiple, identical cis elements or can be used to target multiple TFs using a decoy containing multiple cis elements, each or several targetting a different TF.

During the past decade, the complete genomes of more than 140 different organisms have been sequenced and made available in databases. These databases provide extremely useful collections of organized, validated data, which are indispensable for genomics and proteomics research and the drug discovery process. TFs comprise 6% of the human genome ranking the second position for their abundance and have recently been considered a new class of candidate targets for drug discovery (Roth, 2005). On the other hand, the dODN technology using TFs as molecular targets is emerging as a powerful strategy for gene therapy of broad range of human diseases (Mann and Dzau, 2000; Morishita et al., 2001). Conceptually, our cdODN “one drug-multiple targets” strategy mimics the well-known ‘drug-cocktail’ therapy. Nevertheless, the present “one drug-multiple targets” strategy is devoid of the weaknesses of the ‘drug-cocktail’ therapy involving complicated treatment regimen, undesired drug-drug interactions and increased side effects as well. The cdODN strategy offers resourceful combinations of varying cis-elements for concomitantly targeting multiple molecules in particular biological processes. Here we merely tested the cdODNs potentially applicable to a wide spectrum of cancers since the target oncoproteins NF-κB/E2F/Stat3 are not tissue specific. It is worthy of noting that the cdODN strategy has the potential of targeting specific types of cancers. For example, a cdODN can be designed to treat breast cancer in particular by targeting SNAIL (Martin et al., 2005), ERE (Wang et al., 2003), Brn-3b (Budhram-Mahadeo et al., 1999), SLUG (Tripathi, 2005), and EBS. An ERE (estrogen receptor responsive element) decoy has been shown to be effective in suppressing breast cancer cell growth. The present invention in which multiple ERE sites could be put on one decoy would be predicted to exponentially increase the efficacy of this previously tested decoy. Previous to the instant invention, such a synergy was unexpected. Brn-3b is a repressor of BRCA1 and SLUG is a repressor of BRCA2 (down-regulation and/or mutations of BRCA1/2 have been shown to be critical for breast cancer development). Moreover, the cdODN strategy can also be applied to other disorders in addition to cancer. For instance, TNF-a, GATA-4, FOG-2, and JAK-STAT could be a reasonable set of combination for a cdODN aiming to treat heart failure by reducing apoptosis (Suzuki and Evans, 2004; Kassiri et al., 2005). A cdODN targeting Irx5, Irx3 and Etv1 may be applied to reduce regional heterogeneity of cardiac repolarization so as to minimize arrhythmogenesis since these TFs have been shown to be expressed in transmural gradients across the ventricular wall (Rosati et al., 2006; Costantini et al., 2006) and to be responsible for the transmural difference of a K+channel (Costantini et al., 2006). Therefore, the cdODN technology opens the door to ‘one drug-multiple targets’ intervention, providing promising prototypes of gene therapeutic agents for a wide range of human diseases.

The cdODN technology also opens up new opportunities for creative and rational designs of a variety of combinations integrating varying cis-elements for various purposes and provides an exquisite tool for functional genomics analysis related to identification and characterization of new and known transcription factors and their functions in gene controlling program. It can also be used as a simple and straightforward approach for studying any other biological processes involving multiple factors, multiple genes, multiple signaling pathways, etc.

Although the present invention has been described hereinabove by way of preferred embodiments thereof, it can be modified without departing from the spirit, scope and nature of the subject invention, as defined in the appended claims.

### LIST OF REFERENCES

1. Ahn J D, Kim C H, Magae J, Kim Y H, Kim H J, Park K K, Hong S, Park K G, Lee I K, and Chang Y C (2003) E2F decoy oligodeoxynucleotides effectively inhibit growth of human tumor cells. Biochem Biophys Res Commun 310:1048-1053.
2. Bielinska A, Shivdasani R A, Zhang L, and Nabel G J (1990) Regulation of gene expression with double-stranded phosphorothioate oligonucleotides. Science 250:997-1000.
3. Budhram-Mahadeo V, Ndisang D, Ward T, Weber B L, and Latchman D S (1999) The Brn-3b POU family transcription factor represses expression of the BRCA-1 anti-oncogene in breast cancer cells. Oncogene 18:6684-6691.
4. Chan K S, Sano S, Kiguchi K, Anders J, Komazawa N, Takeda J, and DiGiovanni J (2004) Disruption of Stat3 reveals a critical role in both the initiation and the promotion stages of epithelial carcinogenesis. J Clin Invest 117:720-728.
5. Charpentier G (2002). Oral combination therapy for type 2 diabetes. Diabetes Metab Res Rev 18 (Suppl 3):S70-S76.
6. Costantini D L, Arruda E P, Agarwal P, Kim K H, Zhu Y, Zhu W, Lebel M, Cheng C W, Park C Y, Pierce S A, et al. (2006) The homeodomain transcription factor Irx5 establishes the mouse cardiac ventricular repolarization gradient. Cell 123:347-358.
7. Dolcet X, Llobet D, Pallares J, and Matias-Guiu X (2005) NF-kB in development and progression of human cancer. Virchows Arch 446:475-482.
8. Duyao M P, Kessler D J, Spicer D B, Bartholomew C, Cleveland J L, Siekevitz M, and Sonenshein G E (1992) Transactivation of the c-myc promoter by human T cell leukemia virus type 1 tax is mediated by NF kappa B. J Biol Chem 267:16288-16291.
9. Fry C J, Pearson A, Malinowski E, Bartley S M, Greenblatt J, and Farnham P J (1999) Activation of the murine dihydrofolate reductase promoter by E2F1. A requirement for CBP recruitment. J Biol Chem 274:15883-15891.
10. Henkel J (1999) Attacking AIDS with a ‘cocktail’ therapy? FDA Consum 33:12-17.
11. Ji Y B, Gao S Y, Ji H R, Kong Q, Zhang X J, and Yang B F (2004) Anti-neoplastic efficacy of Haimiding on gastric carcinoma and its mechanisms. World J Gastroenterol 10:484-490.
12. Kandimalla E R, Bhagat L, Cong Y P, Pandey R K, Yu D, Zhao Q, and Agrawal S (2003) Secondary structures in CpG oligonucleotides affect immunostimulatory activity. Biochem Biophys Res Commun 306:948-953.
13. Kassiri Z, Oudit G Y, Sanchez O, Dawood F, Mohammed F F, Nuttall R K, Edwards D R, Liu P P, Backx P H, and Khokha R (2005) Combination of tumor necrosis factor-alpha ablation and matrix metalloproteinase inhibition prevents heart failure after pressure overload in tissue inhibitor of metalloproteinase-3 knock-out mice. Circ Res 97:380-390.
14. Konlee M T H (1998) An evaluation of drug cocktail combinations for their immunological value in preventing/remitting opportunistic infections. HTPosit Health NewsTH 16:2-4.
15. Kreuz S, Siegmund D, Scheurich P, and Wajant H (2001) NFkappaB inducers upregulate cFLIP, a cycloheximide-sensitive inhibitor of death receptor signaling. Mol Cell Biol 21:3964-3973
16. Kumar P (2005) Combination treatment significantly enhances the efficacy of antitumor therapy by preferentially targeting angiogenesis. Lab Invest 85:756-767.
17. La Rosa F A, Pierce J W, and Sonenshein G E (1994) Differential regulation of the c-myc oncogene promoter by the NF-kappa B rel family of transcription factors. Mol Cell Biol 14:1039-1044.

18. Leong P L, Andrews G A, Johnson D E, Dyer K F, Xi S, Mai J C, Robbins P D, Gadiparthi S, Burke N A, Watkins S F, et al. (2004) Targeted inhibition of Stat3 with a decoy oligonucleotide abrogates head and neck cancer cell growth. Proc Nati Acad Sci USA 100:4138-4143.
19. Li C, Zhang F, Lin M, and Liu J. (2004) Induction of S10A9 gene expression by cytokine oncostatin M in breast cancer cells through the STAT3 signaling cascade. Breast Cancer Res Treat 87:123-134.

20. Lin Q, Lai R, Chirieac L R, Li C, Thomazy V A, Grammatikakis I, Rassidakis G Z, Zhang W, Fujio Y, Kunisada K, Hamilton SR, and Amin H M (2005a) Constitutive activation of JAK3/STAT3 in colon carcinoma tumors and cell lines: inhibition of JAK3/STAT3 signaling induces apoptosis and cell cycle arrest of colon carcinoma cells. Am J Pathol 167:969-980.
21. Lin L M, Li B X, Xiao J B, Lin D H, and Yang B F (2005b) Synergistic effect of all-trans-retinoic acid and arsenic trioxide on growth inhibition and apoptosis in human hepatoma, breast cancer, and lung cancer cells in vitro. World J Gastroenterol 11:5633-5637.
22. Lun M, Zhang P L, Siegelmann-Danieli N, Blasick T M, and Brown R E. (2005) Intracellular inhibitory effects of Velcade correlate with morphoproteomic expression of phosphorylated-nuclear factor-kappaB and p53 in breast cancer cell lines. Ann Clin Lab Sci 35:15-24.
23. Mann M J and Dzau V J (2000) Therapeutic applications of transcription factor decoy oligonucleotides. J Clin Invest 106:1071-1075.
24. Mann M J, Whittemore A D, Donaldson M C, Belkin M, Conte M S, Polak J F, Orav E J, Ehsan A, Dell'Acqua G, and Dzau V J (1999) Ex-vivo gene therapy of human vascular bypass grafts with E2F decoy: the PREVENT single-centre, randomized, controlled trial. Lancet 354:1493-1498.
25. Martin T A, Goyal A, Watkins G, and Jiang W G (2005) Expression of the transcription factors snail, slug, and twist and their clinical significance in human breast cancer. Ann Surg Oncol 12:488-496.
26. Micheau O, Lens S, Gaide O, Alevizopoulos K, and Tschopp J (2001) NF-kappaB signals induce the expression of c-FLIP. Mol Cell Biol 21:5299-5305
27. Morishita R, Aoki M, and Kaneda, Y (2001) Decoy oligodeoxynucleotides as novel cardiovascular drugs for cardiovascular disease. Ann NY Acad Sci 947:294-301.
28. Morishita R, Gibbons G H, Horiuchi M, Ellison K E, Nakama M, Zhang L, Kaneda Y, Ogihara T, and Dzau V J (1995) A gene therapy strategy using a transcription factor decoy of the E2F binding site inhibits smooth muscle proliferation in vivo. Proc Nati Acad Sci USA 92:5855-5859.
29. Morishita R, Sugimoto T, Aoki M, Kida I, Tomita N, Moriguchi A, Maeda K, Sawa Y, Kaneda Y, Higaki J, et al. (1997) In vivo transfection of cis element “decoy” against nuclear factor-kappaB binding site prevents myocardial infarction. Nat Med 13:894-899.
30. Nabholtz J M and Gligorov J (2005) Docetaxel/trastuzumab combination therapy for the treatment of breast cancer. Expert Opin Pharmacother 6:1555-1564.
31. Nielsen M, Kaestel C G, Eriksen K W, Woetmann A, Stokkedal T, Kaltoft K, Geisler C, Ropke C, and Odum N (1999) Inhibition of constitutively activated Stat3 correlates with altered Bcl-2/Bax expression and induction of apoptosis in mycosis fungoides tumor cells. Leukemia 13:735-738.
32. Niu G, Wright K L, Ma Y, Wright G M, Huang M, Irby R, Briggs J, Karras J, Cress W D, Pardoll D, et al. (2005) Role of Stat3 in regulating p53 expression and function. Mol Cell Biol 25:7432-7440.
33. Ogihara T (2003) The combination therapy of hypertension to prevent cardiovascular events (COPE) trial: rationale and design. Hypertens Res 28:331-338
34. Pang L, Koren G, Wang Z, and Nattel S (2003) Tissue-specific expression of two human Cav1.2 isoforms under the control of distinct 5′ flanking regulatory elements. FEBS Lett 546:349-354.
35. Park K K, Rue S W, Lee I S, Kim H C, Lee I K, Ahn J D, Kim H S, Yu T S, Kwak J Y, Heintz N H, et al. (2003) Modulation of Sp1-dependent transcription by a cis-acting E2F element in DHFR promoter. Biochem Biophys Res Commun 306:239-43.
36. Roth M (2005) Transcription factors: Are they a real target for future therapeutic strategies? Pharmacologyonline 1:45-66.
37. Rosati B, Grau F, and McKinnon D (2006) Regional variation in mRNA transcript abundance within the ventricular wall. J Mol Cell Cardiol 40:295-302.
38. Sato Y, Miyata M, Sato Y, Nishimaki T, Kochi H, and Kasukawa R (1999) CpG motif-containing DNA fragments from sera of patients with systemic lupus erythematosus proliferate mononuclear cells in vitro. J Rheumatol 26:294-301.
39. Stanelle J, Stiewe T, Rodicker F, Kohler K, Theseling C, and Putzer B M (2003) Mechanism of E2F1-induced apoptosis in primary vascular smooth muscle cells. Cardiovasc Res 59:512-519.
40. Suzuki Y J and Evans T (2004) Regulation of cardiac myocyte apoptosis by the GATA-4 transcription factor. Life Sci 74:1829-1838
41. Tripathi M K (2005) Regulation of BRCA2 gene expression by the SLUG repressor protein in human breast cells. J Biol Chem 280:17163-7171.
42. Wang H, Zhang Y, Cao L, Han H, Wang J, Yang B, Nattel S, and Wang Z (2002) HERG K+ channel: A regulator of tumor cell apoptosis and proliferation. Cancer Res 62:4843-4848.
43. Wang L H, Yang X Y, Zhang X, Mihalic K, Xiao W, and Farrar W L (2003) The cis decoy against the estrogen response element suppresses breast cancer cells via target disrupting c-fos not mitogen-activated protein kinase activity. Cancer Res 63:2046-2051.
44. Xi S, Gooding W E, and Grandis J R (2005) In vivo antitumor efficacy of STAT3 blockade using a transcription factor decoy approach: implications for cancer therapy. Oncogene 24:970-979.
45. Yasui K, Okamoto H, Arii S, and Inazawa J (2003) Association of over-expressed TFDP1 with progression of hepatocellular carcinomas. J Hum Genet 48:609-613.
46. Yokoyama T H, Nakano H, Yamazaki T, Tamai K, Hanada K, and Takahashi G (2005) Enhancement of ultraviolet-induced apoptosis by NF-kappaB decoy oligonucleotides. Br J Dermatol 153 (Suppl 2):47-51.
   All references cited and/or discussed in this specification are incorporated herein by reference in their entirety and to the same extent as if each reference was individually incorporated by reference.

1. A double-stranded decoy oligodeoxynucleotide, said decoy oligodeoxynucleotide comprising a first strand of deoxynucleotides and a second strand of deoxynucleotides substantially complementary to said first strand of deoxynucleotides, said decoy oligodeoxynucleotide comprising at least two cis elements, each of said cis elements specifically targeting a transcription factor.

2. The decoy oligodeoxynucleotide as defined in claim 1, wherein said cis elements all target specifically the same transcription factor.

3. The decoy oligodeoxynucleotide as defined in claim 2, wherein said cis elements are substantially identical.

4. The decoy oligodeoxynucleotide as defined in claim 1, wherein said cis elements target specifically different transcription factors.

5. The decoy oligodeoxynucleotide as defined in claim 1, wherein said cis elements target specifically at least one of transcription factors NF-κB, E2F and Stat3.

6. The decoy oligodeoxynucleotide as defined in claim 1, wherein said cis elements target specifically and simultaneously at least two transcription factors selected from the group consisting of NF-κB, E2F, Stat3, SNAIL, a transcription factor targeting the estrogen receptor responsive element, Brn-3b, SLUG and Ets-binding sites.

7. The decoy oligodeoxynucleotide as defined in claim 1, wherein said cis elements target specifically and simultaneously at least two transcription factors selected from the group consisting of: tumor necrosis factor-alpha, GATA-4, FOG-2 and Janus tyrosine kinase-signal transducer and activator of transcription.

8. The decoy oligodeoxynucleotide as defined in claim 1, wherein said cis elements target specifically and simultaneously at least two transcription factors selected from the group consisting of: Irx5, Irx3 and Etv1.

9. The decoy oligodeoxynucleotide as defined in claim 1, wherein one of said cis elements is comprised in said first strand of deoxynucleotides and another one of said cis elements is comprised in said second strand of deoxynucleotides.

10. The decoy oligodeoxynucleotide as defined in claim 1, wherein one of said first and second strands of deoxynucleotides has the sequence set forth in one of SEQ ID NOs: 4 to 13.

11. The decoy oligodeoxynucleotide as defined in claim 1, wherein at least one of said cis elements has the sequence set forth in one of SEQ ID NOs: 1 to 3.

12. The decoy oligodeoxynucleotide as defined in claim 1, comprising at least three cis elements, said at least three cis elements having respectively the sequences set forth in SEQ IDs NOs: 1, 2 and 3.

13. A method for treating or preventing a disease in a mammal, said disease resulting from the increased presence of a specific transcription factor, said method comprising the administration to said mammal of a therapeutically effective amount of a decoy oligodeoxynucleotide, said decoy oligodeoxynucleotide comprising at least two cis elements, each of said cis elements specifically targeting a respective transcription factor, at least one of said cis elements specifically targeting said specific transcription factor.

14. The method as defined in claim 13, wherein said decoy oligodeoxynucleotide is carried in a pharmaceutically acceptable carrier.

15. The method as defined in claim 13, wherein said disease is cancer.

16. The method as defined in claim 15, wherein said cancer is breast cancer or lung cancer.

17. The method as defined in claim 15, wherein said method results in an inhibition in the growth of a tumor.

18. The method as defined in claim 15, wherein said cancer includes cancer cells from a cell line selected from the group consisting of breast cancel cell line SK-BR-3, breast cancer cell line MCF-7, and lung cancer cell line A549.

19. The method as defined in claim 13, wherein said disease is heart failure or arrhythmia.

20. The method as defined in claim 13, wherein said decoy oligodeoxynucleotide is a decoy ologinucleotide comprising a first strand of deoxynucleotides and a second strand of deoxynucleotides substantially complementary to said first strand of deoxynucleotides, said decoy oligodeoxynucleotide comprising at least two cis elements, each of said cis elements specifically targeting a transcription factor, one of said first and second strands of deoxynucleotides having the sequence set forth in one of SEQ ID NOs: 1 to 13.

21. A method for reducing tumour growth in a mammal, said method comprising the administration to said mammal of a therapeutically effective amount of a substance simultaneously reducing the expression of transcription factors NF-κB, E2F and Stat3.

22. The method as defined in claim 21, wherein said substance is administered intra-tumorally.

23. The method as defined in claim 21, wherein said substance includes a decoy oligodeoxynucleotide comprising a first strand of deoxynucleotides and a second strand of deoxynucleotides substantially complementary to said first strand of deoxynucleotides and bound thereto, said decoy oligodeoxynucleotide comprising at least three cis elements, said at least three cis elements each specifically targeting a respective transcription factor selected from transcription factors NF-κB, E2F and Stat3.

24. The method as defined in claim 23, wherein said at least three cis elements have respectively the sequences set forth in SEQ IDs NOs: 1, 2 and 3

25. The method as defined in claim 23, wherein said decoy oligodeoxynucleotide is carried in a pharmaceutically acceptable carrier.

26. A method for interfering with the expression of at least two genes in mammalian cells, the expression of each of said at least two genes being regulated by a respective transcription factor, said method comprising the transfection in said cells of a decoy oligodeoxynucleotide, said decoy oligodeoxynucleotide comprising at least two cis elements, each of said cis elements specifically targeting one of said respective transcription factors, said decoy oligodeoxynucleotide being administered in an amount effective for substantially reducing the regulatory action of each of said transcription factors.

27. The method as defined in claim 26, wherein each of said cis element is identical with a target core sequence of said respective transcription factor targeted by said cis element.

28. A method as defined in claim 26, wherein each of said cis elements is between 8 and 9 bases long.

29. The method as defined in claim 23, wherein at least one of said cis elements has sequence selected from the sequences set forth in SEQ ID NOs: 1, 2 and 3.

30. (canceled)

31. The method as defined in claim 13, wherein said subject is a human.
